(19) United States
(12) Patent Application Publication
(10) Pub. No.: US 2002/0004477 A1
(43) Pub. Date: Jan. 10, 2002

(54) Therapeutic administration of a modified alpha1 proteinase inhibitor

(76) Inventor: Cynthia L. Bristow, New York, NY (US)

Correspondence Address: LIEBERMAN & NOWAK LLP, 350 FIFTH AVE., SUITE 7412, NEW YORK, NY, 10118, US

(21) Appl. No.: 09/899,500

(22) Filed: Jul. 5, 2001

Related U.S. Application Data
(60) Provisional application No. 60/215,987, filed on Jul. 5, 2000.

(51) Int. Cl. A61K 31/00

(52) U.S. Cl. 514/1

(57) ABSTRACT

A method for the treatment of asymtomatic HIV seropositive individuals to inhibit onset of an AIDS infection and, for suppression of propagation of HIV infections in individuals with a fully developed AIDS infection. The method consists of administering a therapeutic effective amount of a modified \acute{α}₁Proteinase Inhibitor (m\acute{α}₁PI) to an individual within the effected population so as to inhibit and/or prevent \acute{α} PI facilitated HIV entry into healthy/uninfected cells.

### RELATED APPLICATIONS

This application claimsthe filing date of Provisional Patent Application No. 60/215,987 filed Jul. 5, 2000.

### BACKGROUND OF THE INVENTION

1. Field of the Invention

This invention relates to a method and to a composition of matter suitable for use in this method. More specifically, this invention relates to the treatment of asymtomatic HIV seropositive individuals with a modified form of (\acute{α}₁proteinase inhibitor (6,PI) as a means for inhibition of HIV replication in asymtomatic HIV seropositive individuals; and, as a means for suppression of propagation of HIV infections in individuals with a fully developed AIDS infection.

2. Description of the Prior Art

HIV-1 is the name given to a group of highly related viruses which have been identified as the primary etiologic agent of the acquired immunodeficiency syndrome (AIDS) and AIDS related condition (ARC) in humans. HIV-1, also known as HTLV-III, LAV and ARV, is a major worldwide health problem. HIV is a relatively complex retrovirus containing at least seven genes. The viral structural genes designated gag, pol and env respectively code for the viral core proteins, reverse transcriptase, and the viral glycoproteins of the viral envelope. The other HIV genes are accessory genes involved in viral replication. The gag and env genes encode polyproteins, i.e., the proteins synthesized from each of these genes are post-translationally cleaved into several smaller proteins. Previous studies have shown that the proteins coded by the gag and especially the env regions of the HIV genome are immunologically important, since antibodies to the products of the gag and env genes are found in the sera of patients infected with HIV. The env gene encodes a glycoprotein (gp160) with an apparent molecular weight (Mw) of about 160,000 Daltons which is post-translationally cleaved into two glycoproteins, gp120 and gp41, of Mw 120,000 and 41,000 Daltons, respectively. Glycoprotein gpl20 is the external protein of the viral envelope, while gp41 is a transmembrane protein. The gp120 protein associates noncovalently with gp41 such that gp120 is exposed on the cell surface. Both gp120 and gp41 are immunogenic, with antibodies to the proteins readily detectable in sera obtained from patients infected with HIV but who are asymptomatic, and ARC and AIDS patients. The HIV coat protein gp120 binds to the T cell surface protein CD4, also known as the HIV receptor protein. The gp120 protein is crucial to HIV infection, because gp120 binds to CD4 with high affinity to effect HIV entry into the cell. Cells infected by HIV express a cell surface form of gp120 and also shed a soluble form of gp120 protein from their surface. This extracellular, soluble gp120 binds to CD4 on uninfected cclls with high affinity and appears to induce cell death by an unknown mechanism. It is characteristic of HIV infection that infected cells may not lyse, consequently cells expressing gp120 may survive for extended periods of time, acting as reservoirs of the virus and gp120. HIV infection is largely dependent on gp120 binding to CD4 as underscored by the finding that soluble recombinant CD4 lacking its transmembrane and cytosolic sequences can block HIV infectivity and soluble gp120 mediated cell killing. The soluble CD4 also binds to HIV infected cells and soluble gp120. Smith et al., Blocking of HIV-1 Infectivity by a Soluble, Secreted Form ofthe CD4 Antigen, Science, 238: 1704-1706 (1987). The use of soluble CD4 as a therapeutic agent is expensive, however, and there are problems with delivery and stability. Furthermore, CD4 treatment can result in the formation of antibodies specific to CD4 with subsequent autoimmune disease. Weber, Blocks on the Viral Exit, Nature, 345: 573-574 (1990). HIV infection is also thought to occur by cell-to-cell transmission. This form of infection does not rely on extracellular HIV binding to CD4, and cannot be prevented by agents such as soluble CD4. In order to effect cell-to-cell transmission, an infected cell forms contacts termed “syncytia” with a non-infected cell, thus, enabling direct transmission of the virus. Syncytia are visible under a light microscope and are indicators of HIV infection. Since HIV-1 was first identified as the etiologic agent of AIDS, substantial progress has been made in studies on the virus per se, mechanisms by which the virus causes disease and in the development of diagnostic tests to detect exposure to the virus or infection. Progress in HIV vaccines and therapy has been slow due to the heterogeneous nature of the virus and the lack of suitable animal models. See, e.g., Martin, Fast-Acting Slow Viruses, Nature, 345: 572-573 (1990). While a variety of approaches have been taken to formulate pharmaceutical agents suitable for AIDS therapy, many if not all of the drugs create serious side effects which greatly limit their usefulness as therapeutic agents. One drug target is the HIV protease crucial to virus development. HIV protease is an aspartyl protease and hence can be inhibited by agents such as H-261 (tBoc—His—Pro—Phe—His—Leu.psi. [CHOH-CH.sub.2]Val-Ile-His) (Seq.I.D. No.2) and acetyl-pepstatin. Richards, Inhibition of the Aspartyl Proteinase from HIV-2, FEBS Lett., 253: 214-216 (1989). Unfortunately, inhibitors of aspartyl proteases are non-selective and therefore toxic when used in vivo. In addition, several peptide analogues have been found to inhibit HIV protease. Meek et al., Inhibition of HIV-1 Protease in Infected T-lymphocytes by Synthetic Peptide Analogues, Nature, 343: 90-92 (1990). Recently a two-fold symmetric inhibitor of HIV protease was described. Erickson et al., Design, Activity, and 2.8 ANG. Crystal Structure of a C.sub.2 Symmetric Inhibitor Complexed to HIV-1 Protease, Science, 249: 527-533 (1990). This symmetric inhibitor has a selective activity against HIV-1 protease, it is about 10,000 fold more potent against HIV-1 protease than against related cellular enzymes.

The HIV reverse transcriptase is likewise necessary for effective HIV infection and thus; drugs are being sought which inhibit the enzyme3 s activity. Several nucleoside derivatives have been found to inhibit HIV reverse transcriptase. Foremost among these drugs is azidothymidine (AZT, Zidovidine.RTM.). AZT causes serious side effects, however, such that many patients cannot tolerate its administration. Other nucleoside analogues that inhibit HIV, reverse transcriptase have been found to cause even more serious side effects then does AZT.

Several agents have been found to inhibit binding of HIV to T cells. For instance, a pentapeptide and an octapeptide derived from vasoactive intestinal peptide (VIP) have been found to inhibit HIV infection. Moore et al., In vivo Depression of Lymphocyte Traffic in Sheep by VIP and HIV (AIDS)—Related Peptides, Immunopharmacol., 16: 181-189 (1988). Unfortunately, these peptides have the side effect of mimicking the immunosuppression of HIV infection by inhibiting proliferation of T4 cells and are, thus, useless for therapy. Recently, N-carbomethoxycarbonyl-prolylphenylalanyl benzyl ester (CPF), a derivative ofthe dipeptide prolylphenylalanine, was shown to inhibit HIV-1 infection in vitro. CPF interacts with gp120 and blocks the binding of gp120 to CD4. Finberg et al., Prevention of HIV-1 Infection and Preservation of CD4 Function by the Binding of CPF's to gp120, Science, 249:287-291 (1990). Small peptides have been used in the treatment of measles virus and herpes virus infections. A series of carbobenzoxy (Z) peptides, including Z-D-Pro-D-Phe have been shown to inhibit measles and herpes viruses. Miller et al., Antiviral Activity of Carbobenzoxy Di- and Tripeptides on Measles Virus, Appl. Microbiol., 16:1489-1496 (1968); and Nicolaides et al. Potential Antiviral Agents. Carbobenzoxy Di- and Tripeptides Active Against Measles and Herpes Viruses, J. Med. Chem., 11:74-79 (1968). These compounds interact with the target cell and not with the viral protein.

Not surprisingly, the patent literature is replete with inventions that reportedly address one or more or the mechanism associated with HIV infection, specifically, both the prevention of spread of such viral infection and/or the control of such infection once the individual has become symptomatic.

U.S. Pat. No. 5,883,252 (to Tung, et al issued Mar. 16, 1999) & U.S. Pat. No. 6,127,372 (to Tung, et al issued Oct. 3, 2000) discloses anovel class of compounds, and pharmaceutically acceptable derivatives thereof, that are useful as inhibitors of aspartyl proteases, and in particular, HIV aspartyl protease. The Tung, et al compounds can reportedly be used alone or in combination with other therapeutic or prophylactic agents, such as anti-virals, antibiotics, immunomodulators or vaccines, for the treatment or prophylaxis of viral infection. The Tung, et al compounds are reportedly particularly well suited for inhibiting HIV-1 and HIV-2 protease activity and, consequently, may be advantageously used as anti-viral agents against the HIV-1 and HIV-2 viruses. The preferred compounds of the Tung, et al invention are capable of inhibiting HIV viral replication in human CD.sub.4.sup.+cells including T-cells, monocytic lines including macrophages and dendrocytes and other permissive cells. These compounds are, thus, reportedly useful as therapeutic and prophylactic agents to treat or prevent infection by HIV-1 and related viruses which may result in asymptomatic infection, AIDS-related complex (“ARC”), acquired immunodeficiency syndrome (“AIDS”), or similar disease of the immune system.

U.S. Pat. No. 5,627,035 (to Vahlne, et al. issued May 6, 1997) discloses certain relatively short peptides are able to effectively inhibit HIV infection of host cells and syncytia formation between infected and non-infected cells. The Vahlne, et al. peptides have in common a carboxy terminal dipeptide portion with the amino acid sequence Pro-Gly (PG). The tripeptide Gly-Pro-Gly (GPG) is the preferred species. This tripeptide may be used itself or may be extended amino terminally to form a tetra-, penta-or hexapeptide. The peptides are suitable for therapy in mammals including man infected with HIV and in prevention of HIV infection.

U.S. Pat. No. 5,776,933 (to Gordon, et al.. issued Jul. 7, 1998) discloses novel aminediol compounds, pharmaceutical compositions containing these compounds, and methods of using these compounds in inhibiting retroviral protease, particularly useful in the treatment and/or prevention of HIV infection (AIDS).

Not with standing the advances made to date, there continues to exist a need to improve upon existing therapies by not only making them more effective in their suppression of the viruses responsible for ARC & AIDS, but also to reduce their adverse reactions in the individuals undergoing HIV suppression therapies.

### OBJECTS OF THE INVENTION

It is the object of the invention to remedy the above as well as related deficiencies in the prior art.

More specifically, it is the principle object of this invention to provide a method and composition for the inhibition of infection, and for the suppression of progression of infection of healthy cells by HJV viruses.

It is another object of this invention to provide a method and composition for the inhibition of infection, and the suppression of progression of infection, of healthy cells by HIV viruses by treatment of asymptomatic patients (ARC) and patients with active HIV infections with a modified \acute{α}₁ Proteinase Inhibitor (m\acute{α}₁PI).

It is yet another object of this invention to provide a method and composition for the inhibition of infection and the suppression ofprogression ofinfection ofhealthy cells by HIV viruses by replacement of depleted circulating concentrations 6,Proteinase Inhibitor with a modified \acute{α}₁ Proteinase Inhibitor (m\acute{α}₁PI) as means of inhibition of \acute{α}₁PI facilitated HIV entry into healthy/uninfected cells.

### SUMMARY OF THE INVENTION

The above and related objects are achieved by providing a modified \acute{α}₁Proteinase Inhibitor (m\acute{α}₁PI) suitable for administration to asymptomatic individuals who have been exposed to the HIV virus, and who have not as yet developed active AIDS (also hereinafter “ARC individuals”), and to individuals who have active AIDS (also hereinafter “AIDS individuals”). The amino acids that are referenced herein are designated with a standard three-letter abbreviations followed in parenthesis by the numerical position in the \acute{α}₁PI protein sequence, the first amino acid being amino-terminal Glu(1).

One of two known functions of \acute{α}₁PI is covalent inhibition of proteinases. The amino acids involved in covalent or reversible proteinase inhibition (domain 1) lie between Glu (346) and Pro (369). The second known function of \acute{α}₁PI involves its influence on receptor patching and cell motility. The amino acids involved in receptor patching and cell motility (domain 2) lie between Pro (369) and Pro (382).

The modified \acute{α}₁Proteinase Inhibitor (\acute{α}₁PI) suitable for use in this invention comprises an \acute{α}₁PI wherein the traditional surface-associated receptors are modified by rearrangement, substitution of amino acids and/or by glycosylation of domain (2). The modified \acute{α}₁PI can be composed of the full length protein, modified protein, peptide subcomponent derived from the protein, or modified peptide subcomponent ofthe protein. Modification can include site-directed mutagenesis ofthe \acute{α}₁PI gene, modification of glycosylation, or any combinatorial molecules composed of derivatives of \acute{α}₁PI.

The method of this invention involves (a) initially confirming the existence of an \acute{α}₁Proteinase Inhibitor deficiency in the effected population of individuals that can benefit from this type of replacement therapy, and (b) administering a therapeutic effective amount of a modified \acute{α}₁Proteinase Inhibitor (\acute{α}₁PI) to an individual within the effected population so as to inhibit and/or prevent \acute{α}₁PI facilitated HIV entry into healthy/uninfected cells by increasing the available \acute{α}₁PI capacity to inhibit soluble HLE and thereby enhance covalent or reversible inhibition of proteinase involved in the spread and/or propagation of the viruses for responsible AIDS. As set forth in detail herein, it is believed that this result is accomplished by inhibiting \acute{α}₁PI facilitated attachment of HIV onto plasma membranes of cells, or onto plasma membranes of cells, in which the virus is active and attempting to propagated.

### DESCRIPTION OF THE INVENTION INCLUDING PREFERRED EMBODIMENTS

Traditionally, the primary function for human αlProteinase Inhibitor (\acute{α}₁PI, \acute{α}₁antitripsin) has been thought to be inhibition of serine proteinases. The inventor has recently observed that this same \acute{α}₁PI stimulates plasma membrane events by its specific interaction with plasma membrane-associated receptors. More specifically, it has now been demonstrated that \acute{α}₁PI interacts with cell-surface human leukocyte elastase (HLE) to induce co-patching of HLE, CD4, and chemokine receptors thereby promoting specific cellular responses, (Bristow, C.L., Clin. Diagn. Lab. Immunol., Vol. 8,September 2001.). Alternatively, when pre-complexed with soluble HLE, \acute{α}₁PI interacts with the \acute{α}₂macroglobulin/low density lipoprotein receptor related protein (\acute{α}₂M/LRP). This data, thus, suggests an additional function for \acute{α}₁PI involving cell motility and plasma membrane events leading to discrete cellular functions. Accordingly, by modification of the \acute{α}₁PI domain involved in receptor recognition as taught herein, cellular events involving cell motility and CD4-mediated immune responsiveness are also correspondingly altered.

In the process of the definition of the metes and bounds of this invention, it was determined that \acute{α}₁PI is the proteinase inhibitor exhibiting the greatest concentration in serum, and that the proteinase inhibitor encompassing the broadest spectrum of activity is \acute{α}₂-macroglobulin (\acute{α}₂M). It was also determined that during the asymptomatic category of HIV disease, 100% patients of the HIV seropositive patients manifest deficient levels of active \acute{α}₁PI (Bristow, C.L., Clin. Diagn. Lab. Immunol., Vol. 8, September 2001.). In contrast, \acute{α}₂M levels were not significantly different from normal controls. Moreover, individuals with the inherited form of \acute{α}₁PI deficiency, especially males, are notably susceptible to respiratory infections, and 80%, who survive to adulthood, succumb to respiratory failure between the 4^{th} and 6^{th} decade of life (Beminger, R. W. Alpha 1-antitrypsin. J. Med. Vol. 16:23-99, (1986).). Based upon such observations, the inventor concluded from her data that \acute{α}₁PI deficiency acquired during HIV infection could provide a mechanism for the incidence of attendant infections and dramatically reduced life span. These data also suggested that replacement of \acute{α}₁Pl would be efficacious to prevent the pathophysiologic sequelae of \acute{α}₁Pl deficiency. Ironically, in the same study, the inventor found that increased \acute{α}₁PI was correlated with increased viral load. Accordingly, the inventor concluded that \acute{α}₁PI replacement using native \acute{α}₁PI promotes HIV disease progression; however, that \acute{α}₁PI replacement using an appropriately modified \acute{α}₁PI would provide protection against the pathophysiologic sequelae of \acute{α}₁PI deficiency, while, at the same time, protect against increased viral load. More specifically, it was discovered that \acute{α}₁Pl replacement using a modified Proteinase Inhibitor (m\acute{α}₁PI) was feasible to both co-exist with HIV, specifically, did not propagate the virus nor enable viral attachment to the plasma member of immune system cells, as discussed in greater detail herein.

The \acute{α}₁PI deficiency manifested in the asymptomatic HIV patient population suggests predisposition for opportunistic infections. On the other hand, it is known that HIV replication is facilitated by increasing the concentration of active \acute{α}₁PI in vitro and in vivo. Such observation suggests that the increased \acute{α}₁PI concentration, manifested during the acute phase response to opportunistic infections in the symptomatic pre-AIDS patient population, may be etiologic for increased viral load. What was not known, nor appreciated prior to the instant invention, is that by preserving \acute{α}₁PI capacity to inhibit soluble HLE, the capacity of HIV to attach to and entry into cells is diminished.

Accordingly, the \acute{α}₁Proteinase Inhibitor (\acute{α}₁PI) is re-engineered, in accordance with the teachings of this invention, to prevent \acute{α}₁PI facilitated HIV entry into healthy/uninfected cells by increasing the available \acute{Δ}₁PI capacity to inhibit soluble HLE. More specifically, the \acute{α}₁PI domain that initiates chemotaxis has been identified as a hydrophobic pentapeptide concealed within the C-terminal regions of \acute{α}₁PI adjacent and distinct from the proteinase inhibiting domain. The corresponding pentapeptides found in \acute{α}₁PI, Antithrombin III, C1 esterase inhibitor, plasminogen activator inhibitor type 1, and various other serine proteinase inhibitors, contain a pair of phenylalanine (F) residues that share the motif FXFXX or FXXFX, where X represents the hydrophobic amino acids valine (V), leucine (L), isoleucine (I), or methionine (M). The identification of a pentapeptide having a similar motif within the fusion domain of gp41 (FLGFL, where G=glycine) led to the previous identification of the interaction between gp41 and HLE.

The present invention is based upon the modification of \acute{α}₁PI to allow development of combinatorial proteins with structures having the capacity to efficiently inactivate soluble HLE, without promoting plasma membrane-associated events including HIV entry. Because the cellular response influenced by \acute{α}₁PI involves CD4, administration of therapeutic amounts of this m\acute{α}₁Pl, in accordance with the method of this invention, increases an individuals capacity to inhibit soluble HLE and thereby allow management of pathologic immune responses resulting from HIV infection and many other disease states (e.g. microbial organisms, transplantation, autoimmunity, cancer, etc). Within the context of this invention, the phrase “therapeutic amount” is more or less empirically determined by initial reference to the \acute{α}₁PI level of an ARC or an AIDS individual to whom the m\acute{α}₁PI is to be administered, and thereupon administration of sufficient modified Proteinase Inhibitor (m\acute{α}₁PI) to inhibit/prevent \acute{α}₁PI facilitated HIV entry into healthy/uninfected cells by increasing the available \acute{α}₁PI capacity to more effectively inhibit soluble HLE. It is contemplated the administration of modified Proteinase Inhibitor (m\acute{α}₁PI) shall continue until such time as the infection is in remission. It may also be clinically prudent to place an individual on a sustaining dose, and such decision is generally dictated by clinical factors which are, in turn, dependant upon an individual's response to this therapy. In any event, the m\acute{α}₁PI of this invention, when properly administered, does not evoke an immune nor does its evoke reactions which would require discontinued use.

As briefly noted herein, preliminary to definition of this invention, it was necessary to identify the proteinase inhibitor in serum exhibiting the greatest concentration is \acute{α}₁PI; and, the proteinase inhibitor encompassing the broadest spectrum is \acute{α}₂-macroglobulin (\acute{α}₂M). In her study of HIV seropositive patients, the inventor determined that during the asymptomatic phase of HIV disease, 100% patients manifest deficient levels of active \acute{α}₁PI (Bristow, C.L., Clin. Diagn. Lab. Immunol., Vol. 8, September 2001.). In contrast, \acute{α}₂M levels were not significantly different from normal controls. Individuals with the inherited form of \acute{α}₁PI deficiency, especially males, are notably susceptible to respiratory infections, and 80% who survive to adulthood succumb to respiratory failure between the 4^{th} and 6^{th} decade of life (Beminger, 1986). Based upon such observations, the inventor concluded, and her data suggest, that \acute{α}₁PI deficiency acquired during HIV infection is a contributing, if not the principle, mechanism for the incidence of attendant infections and dramatically reduced life span. These data also suggest that replacement of \acute{α}₁PI would also be useful to prevent the pathophysiologic sequelae of \acute{α}₁PI deficiency. Ironically, in this same study, the inventor found that increased \acute{α}₁PI correlated with increased viral load. Accordingly, the inventor concluded that \acute{α}₁PI replacement using native \acute{α}₁PI promotes HIV disease progression; however, that \acute{α}₁PI replacement, using an appropriately modified \acute{α}₁PI, would provide protection against the pathophysiologic sequelae of \acute{α}₁PI deficiency, while at the same time protecting against increased viral load. More specifically, that \acute{α}₁PI replacement using an appropriately modified \acute{α}₁PI is a feasible, and that such \acute{α}₁PI replacement with a modified form of the \acute{α}₁PI replacement can both co-exist with HIV, and avoid the development AIDS in asymptomatic individuals (ARC Individuals), or propagation of the HIV virus in individual that are symptomatic of AIDS.

The Examples which follow further define, describe and illustrate one or more of the preferred embodiments of this invention. Parts and percentages appearing in such Examples are by weight unless otherwise indicated. Procedures, techniques and equipment utilized in the preparation and evaluation of the compositions of this invention are in accordance in standard techniques and procedures unless specified otherwise.

### EXAMPLES

The therapeutic method of this invention is based upon the use of a modified \acute{α}₁PI to replace depleted circulating concentrations of endogenous \acute{α}₁PI, while at the same time prevent \acute{α}₁PI facilitated HIV entry. As noted briefly herein, the engineering of a modified \acute{α}₁PI for the method of this invention is based upon the fuinctions/properties of \acute{α}₁PI in covalent inhibition of proteinases, and the structure of the of \acute{α}₁PI.

Properties & Structure of \acute{α}₁PI —The amino acids involved in covalent or reversible proteinase inhibition (domain 1) lie between Glu (346) and Pro (369). The second known function of \acute{α}₁PI involves its influence on receptor patching and cell motility. The amino acids involved in receptor patching and cell motility (domain 2) lie between Pro (369) and Pro (382). The contiguous codons for both functional domains are within the fifth exon of the \acute{α}₁PI gene. An \acute{α}₁PI molecule is capable of undergoing glycosylation site at Asn (390). The \acute{α}₁PI is comprised of single polypeptide chain containing 16% carbohydrate. Glycosylation is known to influence intracellular processing and subsequent functional capacities of \acute{α}₁PI. Three different translation initiation sites for the \acute{α}₁PI have been identified. and are designated 5! to 3′ as exons A, B, and C. Exon C is exclusively utilized by hepatocytes. Exon A is utilized by quiescent lymphocytes, and mononuclear phagocytes. When these cells are activated, exon B is utilized. Recombinant \acute{α}₁PI composed of a range of variant constructs are also currently available.

A m\acute{α}₁PI molecule suitable for use in the modulation of cell surface HLE specific peptide component (domain 2) of this molecule by an iterative process of mutagenesis, expression, chromatographic selection, and amplification. In this process, a gene encoding a potential binding domain, is obtained by random mutagenesis of a limited number of predetermined codons, and such gene fused to a genetic element which causes the resulting chimeric expression product to be displayed on the outer surface of a virus (e.g. a filamentous phage) or a cell. Chromatographic selection is then used to identify viruses or cells whose genome includes a fused gene coded for the protein which is bound to the chromatographic target. The foregoing technique for preparation of a modified peptide of this invention is more fully described in Ladner, et al. U.S. Pat. No. 5,571,698, which is herein incorporated by reference in its entirety.

Constructs of Modified \acute{α}₁Proteinase Inhibitor (m\acute{α}₁PI)—As above noted, the objective of the present invention is to modify the interaction of \acute{α}₁PI with surface-associated receptors involving the amino acids and glycosylation of domain 2 of \acute{α}₁PI. The modified \acute{α}₁PI can be composed of the full length protein, modified protein, peptide subcomponent derived from the protein, or modified peptide subcomponent ofthe protein. Modification can include site-directed mutagenesis ofthe \acute{α}₁PI gene, modification of glycosylation, or any combinatorial molecules composed of derivatives of \acute{α}₁PI. Effectiveness of modified \acute{α}₁PI can be measured by detecting proteolytic activity, signal transduction, cellular adherence, cell motility, HIV infectivity, uropod formation, and cell surface density of HLE using isolated cell preparations.

Effectiveness of plasma membrane-associated HLE interactive peptides can be measured in vivo using primate models. Immune cells of rodents have been shown to exhibit a cell surface protein with similar amidolytic activity as HLE, but which was identified as a different protein with different substrate specificity (Sugimura, K., Yamasaki, N., Matsuura, M., and Watanabe, T Antigen-Specific T cell Suppressor Factor (TsF): Isolation of a cDNA Clone Encoding For. Eur. J Immunol Vol. 15:873-880, (1985); Bristow, C. L., Lyford, L. K., Stevens, D. P., and Flood, P.M. Elastase Is a Constituent Product of T-Cells, Biochem Biophys Res Comm. Vol. 181:232-239.(1991).

What is claimed is:

1. A method for the treatment of asymtomatic HIV seropositive individuals to inhibit onset of an AIDS infection and, for suppression of propagation of HIV infections in individuals with a fully developed AIDS infection, said method comprising:
A. Initially testing said individuals for determination of the existence of an \acute{α}₁Proteinase Inhibitor deficiency; and
B. administering to \acute{α}₁Proteinase Inhibitor deficient individuals a therapeutic effective amount of a modified \acute{α}₁Proteinase Inhibitor (m\acute{α}₁PI) sufficient to inhibit and/or prevent \acute{α}₁PI facilitated HIV entry into healthy/uninfected cells,
said modified \acute{α}₁Proteinase Inhibitor (m\acute{α}₁PI) comprising an \acute{α}₁Proteinase Inhibitor (\acute{α}₁PI) having a first functional site that is physiologically involved in covalent or reversible proteinase inhibition (domain 1) that is specific for inhibition of soluble Human Leukocyte Elastase (HLE) and a second functional site that is physiologically involved in cell surface protein CD4 receptor patching and cell motility (domain 2),
a said first functional site being further characterized as a having a protein segment that is positioned between the amino acids Glu (346) and Pro (369) and
said second functional site being further characterized as a having a protein segment that is positioned between the amino acids Pro (369) and Pro (382) , wherein the surface-associated receptors of said second functional site have been modified by rearrangement, substitution of amino acids and/or by glycosylation of said protein segment, so as to inhibit said HIV virus from patching a cell surface protein CD4 receptor, thereby enable management of pathologic immune responses resulting from an HIV infection with said modified \acute{α}₁Proteinase Inhibitor (m\acute{α}₁PI).
